SEQUENCE LISTING (6 sequences)

SEQ ID NO: 1 - protein, 5 aa, Homo sapiens
KKRPK

SEQ ID NO: 2 - protein, 8 aa, Homo sapiens
PHGGGWGQ

SEQ ID NO: 3 - protein, 6 aa, Homo sapiens
KKRPKP

SEQ ID NO: 4 - protein, 7 aa, Homo sapiens
DYKDDDK

SEQ ID NO: 5 - protein, 11 aa, Homo sapiens
KKRPKPGGWNT

SEQ ID NO: 6 - protein, 11 aa, Homo sapiens
GGSRYPGQGSP

(19) United States
(12) Patent Application Publication
(10) Pub. No.: US 2003/0114360 A1
(43) Pub. Date: Jun. 19, 2003

(54) Copolymers and methods of treating prion-related diseases

(76) Inventors: Neil R. Cashman, Toronto (CA); Marc Pinard, Montreal (CA); Harry Ledebur JR., Montreal (CA)

Correspondence Address: CLARK & ELBING LLP, 101 FEDERAL STREET, BOSTON, MA, 02110, US

(21) Appl. No.: 10/316,534

(22) Filed: Dec. 10, 2002

Related U.S. Application Data
(63) Continuation of application No. 09/597,531, filed on Jun. 20, 2000.

(51) Int. Cl. A61K 38/16

(52) U.S. Cl. 514/2

(57) ABSTRACT

The use of a pharmaceutical composition comprising a peptide copolymer of lysine and tyrosine (e.g. copaxone) in combatting prion-related diseases is described.

### CROSS-REFERENCE TO RELATED APPLICATIONS

This application is a continuation of U.S. application Ser. No. 09/597,531, filed Jun. 20, 2000, hereby incorporated by reference.

### BACKGROUND OF THE INVENTION

This invention relates to pharmaceutical compositions, kits and methods and uses of such compositions and kits to combat prion-related and other neurodegenerative diseases in humans and animals such as domestic animals and livestock.

Prion infectivity is associated with accumulation of PrP^{Sc}, a protease-resistant conformational isoform of a normal cellular protein PrP^{C}, and appears unassociated with a nucleic acid component as are conventional infectious agents such as viruses and bacteria (reviewed in Prusiner, Proc. Natl. Acad. Sci. USA 5:3363-83 (1998); Brown et al, Nature 380(6572):345-7 (1996); Cashman, Can. Med. Assoc. J. 157:1381-5 (1997)). The human prion diseases include Creutzfeldt-Jakob disease (CJD), Gerstmann-Sträussler-Scheinker syndrome (GSS), fatal familial insomnia (FFI) and kuru, an epidemic prion disease of the Fore people of New Guinea transmitted by ritual cannibalism.

CJD in developed countries can be considered as 4 separate disease entities: conventional sporadic CJD (˜85% of cases), familial CJD (˜15%), iatrogenic CJD (<1%), and “new variant” CJD (vCJD; more than 50 confirmed cases to date of this filing). Variant CJD is a novel human prion disease, which has emerged in the United Kingdom and continental Europe (Will et al, Lancet 347:921-5 (1996)), is cross-species infection by consumption of cattle tissues contaminated with the infectious agent of bovine spongiform encephalopathy (BSE). The oral-peripheral-neural route of vCJD transmission, and the high peripheral accumulation of PrP^{Sc} in lymphoid tissues of infected individuals (Hill et al, Lancet 349:99-100 (1997)), raises the disturbing spectre of entire populations unknowingly incubating this fatal untreatable disorder. An effective treatment or prophylaxis for vCJD would spare many lives.

WO97/45746 published on Dec. 4, 1997 discloses methods of identifying potential therapeutic compounds for treatment of prion-protein-related disorders by measuring the binding of selected prion-binding protein to scrapie agent protein (PrP^{Sc}) or its normal cellular precursor (PrP^{C}) in the presence or absence of a test compound. Various prion-binding proteins (e.g. cadherins) were identified but this reference does not appear to direct the reader to candidate test compounds.

We have now identified a group of compounds that specifically inhibit a fusion protein of a prion protein (PrP) and alkaline phosphatase (AP), abbreviated as PrP-AP, from binding to cell surfaces. The binding inhibition appears to be specific to PrP rather than a general binding inhibition. The group of compounds is exemplified by the known compound copaxone.

U.S. Pat. No. 3,849,550 (Teitelbaum et al) discloses a composition of matter for use in treatment or prevention of certain autoimmune diseases affecting the brain (e.g. experimental allergic encephalomyelitis (EAE)). The compounds of the composition have a molecular weight range of 15,000 to 25,000 and include what later was referred to as copaxone.

WO95/31990 discloses improved compositions of copolymer-1 (copaxone) substantially free of species having a molecular weight over 40 Kilodaltons. The utility disclosed is as an immunotherapy for multiple sclerosis after it had been observed that myelin basic proteins (MBP) (of which copolymer-1 is a synthetic polypeptide analog) can prevent or arrest EAE which is a disease resembling multiple sclerosis that can be induced in susceptible animals.

WO95/33475 discloses the use of copolymer 1 in the manufacture of a medicament for treatment of visual impairment associated with multiple sclerosis.

The development of copaxone as an innovative drug for treatment of multiple sclerosis is described in Arnon, R., Immunology Letters 50 (1996) 1-15. Teitelbaum, D., et al in Isr. J. Med. Sci. 33 (1997) 280-284 in discussing the progress of copolymer-1 from the laboratory to the FDA for treatment of multiple sclerosis indicate that copolymer-1 is postulated to activate the antigen-specific suppressor T-cell system. WO98/30327 discloses the treatment of multiple sclerosis through ingestion or inhalation of copolymer-1 (copaxone) having an average molecular weight of 4,700 to 11,000 daltons. There does not appear to be any suggestion that any particular group of compounds related to copaxone could have utility in treatment of prion-related diseases or conditions. The need for some means of treatment of such prion-related diseases or conditions is manifest in view of increased profile of such diseases as Creutzfeldt-Jakob disease (CJD) and bovine spongiform encephalopathy (BSE) in recent years.

### SUMMARY OF THE INVENTION

The invention provides a pharmaceutical composition that inhibits binding of prion protein to a cell surface or combats a prion-related disease comprising a positively charged copolymer including a basic amino acid selected from the group consisting of lysine and arginine in association with a pharmaceutically acceptable diluent or carrier.

The invention also provides a pharmaceutical kit for combatting a prion-related disease comprising a positively charged copolymer including a basic amino acid selected from the group consisting of lysine and arginine, a pharmaceutically acceptable diluent or carrier and instructions for use in combatting said disease.

The invention further provides a method of inhibiting binding of a prion protein to a cell surface, preventing or treating a prion-related disease in a mammal which comprises administering a pharmaceutically effective amount of a copolymer of the invention to said mammal.

The invention additionally comprises treating a neurodegenerative condition, an autoimmune condition, a cancer or neoplastic condition or a condition associated with transplantation in a mammal which comprises administering a pharmaceutically effective amount of a copolymer of the invention to said mammal.

The invention additionally provides use of a copolymer of the invention to inhibit binding of a prion protein to a cell surface and combat a prion-related disease.

In preferred features of the invention:

(a) the copolymer additionally comprises at least one amino acid selected from the group consisting of glutamate, alanine, tyrosine, especially glutamate and alanine. Copaxone is particularly preferred;

(b) the copolymer has a molecular weight in the range of from about 2 to 4 to 130 Kilodaltons (Kd). Activity appears to be related to a combination of charge and molecular weight (see on), among other things. Preferred molecular weight ranges include 20-130 Kd, especially 50-130 Kd, through as an alternative molecular weight ranges of 2-40 Kd, especially 2-20 Kd, more particularly 4 to 8 or 11 Kd can be considered, especially for copaxone. By molecular weight range it is understood that at least 75% of its molar fractions the copolymer lies within the molecular weight range specified.

The copolymer of the invention may comprise amino acids lysine (K), arginine (R) and proline (P). Preferred copolymers of the invention are those (a) wherein lysine, arginine and proline are present in the ratio 3 lysine:1 arginine:1 proline; (b) wherein lysine, arginine and proline are present in the ratio 3 lysine:1 arginine:2 proline; (c) wherein said copolymer additionally comprises glycine and lysine, arginine, proline and glycine are present in the ratio 3 lysine:1 arginine:2 proline:1 glycine; (d) wherein said copolymer additionally comprises glycine and lysine, arginine, proline and glycine are present in the ratio 3 lysine:1 arginine:2 proline:2 glycine; (e) wherein said copolymer additionally comprises tryptophan and lysine, arginine, proline, glycine and tryptophan are present in the ratio 3 lysine:1 arginine:2 proline:2 glycine:1 tryptophan; (f) wherein said copolymer additionally comprises asparagine and lysine, arginine, proline, glycine, tryptophan and asparagine are present in the ratio 3 lysine:1 arginine:2 proline:2 glycine:1 tryptophan:1 asparagine; or (g) wherein said copolymer additionally comprises threonine and lysine, arginine, proline, glycine, tryptophan, asparagine and threonine are present in the ratio 3 lysine:1 arginine:2 proline:2 glycine:1 tryptophan:1 asparagine:1 threonine.

The invention also provides a method of modulating cell activators in a mammal comprising administering a cell activation modulatory amount of a copolymer of the invention to said mammal.

The invention additionally provides a method of preventing or treating a prion-related disease in a mammal which comprises administering to said mammal a pharmaceutically acceptable amount of a copolymer of the invention sufficient to inhibit prion recruitment or conversion in said mammal.

Without being limited by theory it appears that the copolymers of the invention compete with prion proteins (PrPs) for prion protein binding proteins (PrPBPs) found on cell surfaces.

By “copolymer” is meant a polymer of dissimilar amino acids. Usually a copolymer can be regarded as a “random” association of amino acids in a defined molar ratio. The copolymers of the invention have positive charge and contain at least one of lysine and arginine. Positive charge is observed at approximately neutral pH values. Preferably the copolymers have a net positive charge. Copolymers of the invention may lack a defined sequence (like copaxone) or may comprise a sequence that is partially or completely defined.

By “prion protein binding protein” or “PrPBP” is meant any protein which binds to a “prion protein” or “PrP” in a saturable and displaceable manner, such as a cadherin. Preferably, this binding is also “high affinity” under normal physiological conditions and conformation-dependent. PrPBPs may also be naturally present (or engineered to be present) all or part of the time in the cytoplasm, cytoplasmic organelles, or nucleus of a host cell. PrPBPs may also be present as a family of proteins in a particular cell, tissue, or organism, and in particular, from mammals such as humans or domesticated animals, for example, sheep, cows, cats, and goats.

By “high affinity” binding is meant an affinity constant (between a prion protein and PrPBP) of less than 100 μM, less than 10 μM, less than 1 μM, less than 100 nM, preferably less than 10 nM, and more preferably less than 2 nM or even 1 nM.

By “saturable” binding is meant binding (between a prion protein and PrPBP) which stops increasing after having reached a certain maximal level, indicating that there are a finite number of binding sites for one of the proteins, and that these binding sites are specific. This is in contrast to the non-specific and continually increasing binding which is characteristic of a protein which adheres non-specifically to cell surfaces.

By “conformation dependent binding” is meant binding that occurs on a non-denatured protein that has been properly post-translationally modified, folded and transported such that its normal physiological binding characteristics remain intact.

By “competitive” binding is meant binding (between a prion protein and PrPBP) which is progressively inhibited by increasing concentrations of an unlabeled form of one of the proteins (for example, PrP).

By “half maximal inhibitory concentration” (IC50) is meant a concentration of a therapeutic of the invention that is sufficient to inhibit binding of half of the PrP-AP present at 1/10 dilution in dose-responsive experiments. In experiments described in detail below copaxone had an IC50 of 0.5 μM. Preferably therapeutics have IC50 values below 5 μM, more preferably below 1 μM, particularly below 0.5 μM, more particularly below 0.1 μM, especially below 0.05 μM, more especially below 0.01 μM.

By “prion diseases” is meant a group of rapidly progressive, fatal, and untreatable brain degenerative disorders including, without limitation, Creutzfeldt-Jakob disease (CJD), Kuru, Gerstmann-Straussler syndrome, and fatal familial insomnia in humans (Prusiner, Science 252:1515-1522 (1991)), scrapie in sheep and goats, and spongiform encephalopathy in cattle, as well as recently described prion disease in other ruminants and cats.

By “treatment of prion diseases” is meant the ability to reduce, prevent, or retard the onset of any symptom associated with prion diseases, particularly those resulting in spongiform change, neuronal cell loss, astrocytic proliferation, accumulation of PrPSc protein, dementia, and death.

By “prion recruitment or conversion” is meant the ability of a PrP^{Sc} isoform to recruit or convert a normal cellular protein PrP^{C} to PrP^{Sc}.

### BRIEF DESCRIPTION OF THE DRAWINGS

FIG. 1 shows the binding pattern of PrP-AP to three test cell lines.

FIG. 2 shows inhibition of PrP-AP binding by a PrP peptide.

FIGS. 3A-3C show inhibition of PrP-AP binding to three test cell lines by copaxone.

FIG. 4 shows that mannitol, a carrier used with copaxone, takes no part in inhibiting PrP-AP binding.

FIG. 5 shows a dose-response curve for copaxone inhibition of PrP-AP binding.

FIG. 6 shows that copaxone has no effect on ¹²⁵I-epidermal growth factor (EGF) binding to H4 cells.

FIG. 7 shows that copaxone has no effect on biotinylated transferrin binding to H4 cells.

FIG. 8 shows other polymers (p1152, p1278, p4149) as well as copaxone inhibit PrP-AP binding to H4 cells.

FIG. 9 shows that some copolymers that have more negative charges do not inhibit PrP-AP binding.

FIGS. 10A and 10B show that other copolymers tested have no effect on 125I-EGF or biotinylated transferrin binding.

### DETAILED DESCRIPTION OF THE INVENTION

1. Binding of PrP to its Cell Surface Receptor (in cis or trans) is Dependent Upon the N-Terminus of PrP.

The mature human prion protein appears to comprise two very different structural domains (Riek et al, Proc. Natl. Acad. Sci. USA 95(20):11667-72 (1998); Liu et al, Biochemistry 38(17):5362-77 (1999); Zahn et al, Proc. Natl. Acad. Sci. USA 97 (2000)): an unstructured region (codons 23 through approximately 120), and a structured domain containing 3 alpha helices and one short beta sheet, in addition to loops connecting these regions of secondary structure (codons 120-231). The unstructured N-terminus contains a short region which is rich in basic amino acids (codons 23-50), particularly at the very N-terminus (KKRPK, SEQ ID NO: 1), and a region (codons 51-90) which contains four perfect, and one imperfect, octapeptide repeats (PHGGGWGQ, SEQ ID NO: 2) which have been found to bind copper at the cell surface (Brown et al, Ann. Neurol. 20:597-602 (1986); Stockel et al, Biochemistry 37:7185-93 (1998); Viles et al, Proc. Natl. Acad. Sci. USA 96(5):2042-7 (1999)). Despite being unstructured in NMR studies, the N-terminal domain does exert some impact on the conformation of the structured domain, as has been recently reported by Wuthrich and colleagues (Zahn et al op. cit.). Moreover, the binding of copper to the octapeptide repeats has been found to confer structure on the N-terminus, which is apparently transduced to the structured domain (Stockel et al, op. cit.).

An interaction between PrP and its receptor may occur in trans (on adjacent but opposing cells in which a PrP-receptor interaction might be involved in adhesion or signal transduction) or cis (on the same cell surface, potentially involved in trafficking to membrane domains for endocytosis or for cell domain-specific signaling). We anticipate that the freely mobile unstructured region of PrP^{C} would be better situated to interact with a receptor protein at the cell surface than the globular structured region of the molecule. We also anticipate that binding of the N-terminus to its receptor may, like copper, exert some effect on the structured domain which may impact upon the conformational changes underlying prion protein conversion.

In support of these conjectures, Harris and colleagues (Shyng et al, J. Biol. Chem. 274(24):14793-800 (1995)) have found that an intact N-terminus is essential for endocytosis of chicken or mouse PrP^{C} expressed by mouse neuroblastoma cells, consistent with the notion that an N-terminal domain of PrP^{C} (a glycosyl phosphatidylinositol-anchored protein) interacts in cis with a transmembrane protein to be trafficked to coated pits. Harris and colleagues have later reported that copper stimulates endocytosis of PrP^{C}, also implicating the N-terminus in this function (Pauly and Harris, J. Biol. Chem. 273(50):33107-10 (1998)).

Using PrP-AP as a probe for the prion cell surface receptor, we have observed that the integrity of the prion N-terminus is essential for binding, in that protease cleavage of the N-terminal 10-20 amino acids ablates cell surface binding of PrP-AP (see WO97/45746). Moreover, peptides synthesized from the N-terminal sequence of the mature prion protein can inhibit binding of PrP-AP to the cell surface (see again WO97/45746). Although it is clear that an intact N-terminus is absolutely essential for cell surface binding, it is not clear at present whether the N-terminus itself is responsible in itself for the binding interaction, or whether the N-terminus interacts with other portions of the prion protein to allow the true binding site to become accessible for binding.

2. Conversion of PrP^{C} to PrPSc is Dependent Upon an Intact N-Terminus.

In the “prion recruitment reaction”, predominantly alpha-helical PrP^{C} undergoes a structural conversion to predominantly beta-sheet PrP^{Sc}, in a reaction catalyzed by PrP^{Sc}. The conversion may occur by a template-driven process in which PrP^{Sc} functions as a “conformase” (Prusiner, Proc. Natl. Acad. Sci. USA 5:3363-83 (1998)), or by a seeded polymerization process in which monomeric prPc is structurally converted as it is aggregated into polymeric PrP^{Sc} (Lansbury and Caughey, Chem. Biol. 2(1):1-5 (1995)). It is also possible that features of both models apply, or some other currently unknown process. Conversion of PrP^{C} to PrP^{Sc} is accompanied by marked changes in the physicochemical properties of the molecule, including resistance to protease digestion, and decreased solubility in non-denaturing detergents.

Caughey and colleagues have pioneered prion conversion in vitro, finding that the protease-resistant fragment of PrP^{Sc} designated PrP27-30 is adequate to convert PrP^{C} to protease resistant state (Kocisko et al, Nature 370(6489):471-4 (1994)). This process is dependent on two phases: a phase in which PrP^{C} binds to PrP27-30, and a phase in which the conversion to a protease-resistant isoform occurs (Horiuchi and Caughey, EMBO J. 18(12):3193-203 (1999)). As PrP27-30 lacks the N-terminal ˜90 amino acids of the unstructured domain, it could be construed from this work that the N-terminus is not required for the process of prion conversion. However, this group has been unable to identify increased infectivity generated from the in vitro conversion reaction, as would be expected if the conversion was a bona fide measure of the prion infectious process. These data are consistent with the possibility that the C-terminal structured region may not contain the sole determinants of prion conversion.

Notably, some genetic evidence supports a role for the N-terminus in human prion diseases. Spontaneous conversion of PrP^{C} to PrP^{Sc} is accelerated by prion protein gene mutations associated with the familial prion diseases. Mutations associated with familial prion disease can be considered in two groupings: point mutations are found in the structured region of the protein, and expansion mutations of the N-terminal octapeptide repeats (reviewed by Ridley et al, Dev. Biol. Stand. 80:15-23 (1993)). It is thus reasonable to posit that both regions of the molecule may participate in the conversion of PrP^{C} to PrP^{Sc}, implicating a role for the receptor-binding N-terminal region in the propagation of infectivity.

Transgenic mouse experiments also provide evidence for a role of the prion N-terminus in propagation of prion infectivity. In Tg(MoPrP, D23-88)Prnp^{0/0} transgenic mice constructed by Prusiner and colleagues (Supattapone et al, Proc. Natl. Acad. Sci. USA 96:14529-341 (1999)), deletion of amino acids 23-88 (approximating the entire N-terminal unstructured region) on a PrP−/− background, experimental scrapie with RML prions cannot be transmitted by intracerebral inoculation. However, Weissmann and colleagues have shown that similar deletions retaining the sequence of the N-terminal 10 amino acids [Tg(MoPrP, D32-80)Prnp^{0/0} and Tg(MoPrP, D32-92) Prnp^{0/0}] are capable of propagating infectivity (Fischer et al, EMBO J. 15(6):1255-64 (1996); Shmerling et al, Cell 93(2):203-14 (1998)). This constitutes direct evidence for a critical role in prion conversion of the N-terminal basic-rich domain which we have identified as key to binding of PrP to its cell surface receptor (see above). Moreover, experiments performed by Prusiner and colleagues (Supattapone et al, op. cit.) show that the co-expression of full-length wild-type PrP^{C} in Tg(MoPrP, D23-88)Prnp^{0/0} mice can overcome the transmission barrier of the N-terminal deletion mutant, suggesting that the binding of prion proteins to their receptor can enhance the conversion of prion proteins lacking binding domains in a “lateral” process mediated by protein contact through the structured domain of the molecule. A similar enhancement of acquired protease resistance upon prion proteins possessing intact N-terminus has been observed by Prusiner and colleagues (Supattapone et al, op. cit.) in vitro in scrapie-infected neuroblastoma cells (ScN2a cells). Finally, Prusiner and colleagues have recently published a study demonstrating that dominant-negative inhibition of PrP^{Sc} formation is partially dependent on the very N-terminus of the mature prion protein, comprising the amino acids KKRPKP (SEQ ID NO: 3) (Zulianello et al, J. Virol. 74(9):4351-60 (2000)).

Prusiner and colleagues (Telling et al, Protein Science 6(4):825-833 (1997)) have also shown that construction of transgenic mice expressing PrP tagged at the N-terminus with the “FLAG peptide” (DYKDDDDK, SEQ ID NO: 4) accelerated scrapie incubation times, whereas more proximal placement of the FLAG peptide did not, again consistent with a role for the prion protein N-terminus in the propagation of infectivity.

Prusiner et al, J. Virol. 74(9):4351-60 (2000) discuss dominant-negative inhibition of prion formation being diminished by deletion mutagenesis of the prion protein.

3. Agents that Block the Interaction of PrP with its Cell Surface Receptor will be Effective in the Prophylaxis and Treatment of Prion Disease.

Given our findings that the PrP N-terminus is essential for binding of PrP to the cell surface, and the findings of other laboratories that the N-terminus participates in the conversion of PrP^{C} to PrP^{Sc} in vivo experimental models, it should follow that inhibitors of PrP-receptor interaction will inhibit prion recruitment. Several mechanisms can be proposed for inhibition of recruitment. Binding of PrP^{C} to its receptor may bring PrP^{C} molecules in direct contact with PrP^{Sc}, a requirement for conversion in vitro; inhibition by exogenous compounds of “oligomerization” at the cell surface may block this contact. Another possible mechanism of action for inhibitors of PrP binding upon infectivity might be that the binding of PrP^{C} to its receptor induces a conformational change, or may constrain plasticity, that promotes conversion; inhibition of receptor binding may block this process. Finally binding of PrP at the cell surface may affect its internalization and processing to PrP^{Sc}, a process may occur in an intracellular compartment such as endosomes and lysosomes.

There are few data in the literature supporting the hypothesis that agents which compete with the binding interaction of PrP and its receptor(s) would be effective in the prophylaxis and treatment of prion disease. A number of negatively charged compounds studied by Caughey and colleagues (Demaimay et al, J. Neurochem. 71(6):2534-41 (1998)) have been shown to attenuate prion propagation in vitro and in vivo, such as congo red. Prusiner and colleagues (Supattapone et al, Cell 96:869-878 (1999)) have found that positively charged branched polyamines and derivatives can “cure” certain infected neuroblastoma cells lines of scrapie infection. Although the effect of these charged compounds has been attributed by these investigators to interaction with the structured domain of PrP27-30, an alternative possibility is that these compounds compete the charge interaction between the positively changed N-terminus of PrP and its receptor binding site, which would be expected to contain negatively charged residues.

We hypothesized that compounds containing high content of positive charge would be effective competitors of the binding of the prion protein to its cell surface receptor, as binding of PrP-AP is critically dependent on an intact N-terminus, and binding can be efficiently competed with a N-terminal 11-mer rich in basic amino acids. Copaxone and related copolymers have been developed to mimic charge attributes of myelin basic protein, and can ameliorate experimental encephalomyelitis and multiple sclerosis, autoimmune diseases directed against CNS myelin. We find that copaxone and related copolymers strongly inhibit the interaction of PrP with its cell surface receptor, which is dependent upon molecular weight and content of basic amino acids. We contend that administration of copaxone or related copolymers would be effective in prophylaxis or treatment of Creutzfeldt-Jakob Disease, familial and iatrogenic prion diseases, and variant CJD. Indeed, it is possible that administration of copaxone to subjects peripherally incubated VCJD may help extinguish propagation of infectivity and prevent neurological symptomatology from emerging. The safety and tolerability of copaxone would make widespread prophylaxis a feasible undertaking. As mechanisms of cell death may be conserved between prion diseases and other neurodegenerative diseases, it is possible that copaxone and other copolymers might be effective in the treatment of Alzheimer's disease, Parkinson's disease, amyotrophic lateral sclerosis, and other CNS disorders.

4. Inhibition of the Interaction of PrP with its Cell Surface Receptor will Have Efficacy in Disorders of Uncontrolled Proliferation, and Abnormal or Undesirable Cell Activation.

The normal cellular isoform of the prion protein appears to possess function, although its means of signal transduction is not clear. For proliferation incompetent cells such as neurons and muscle fibers, PrP^{C} can be shown to participate in cell death, whereas in proliferation-competent cells such as lymphocytes and astrocytes, PrP^{C} can be shown to participate in cell activation. As prion protein participates in cell activation, agents which block the interaction of PrP with its cell surface receptor will have utility in disorders of uncontrolled proliferation, such as cancer, and abnormal or undesirable cell activation, such as autoimmune disease and transplant rejection.

The prominent neurodegeneration occurring in prion diseases has been shown to require expression of PrP^{C}. Aguzzi and colleagues (Brandner et al, Proc. Natl. Acad. Sci. USA 93(23):13148-51 (1996)) have studied the effects of implanting neural tissue expressing PrP^{C} in mouse PrP −/− knockout brain. When experimental scrapie is induced in these animals by intracranial inoculation, the neural graft displays all the neuropathic features of prion disease, including neuronal degeneration and astrocytic activation. However, surrounding brain not expressing PrP^{C} displays no pathological features of scrapie, despite the presence of PrP^{Sc} that has diffused from the graft. These findings indicate that PrP^{C} is required for expression of scrapie neuropathology, and implicate a role for PrP^{C} in the signaling that leads to neuronal cell death and astrocyte activation.

Kretchmar and colleagues (Brown et al, Nature 390(6661):684-7 (1996)) have observed a similar role for PrP^{C} in vitro. Activation of microglia by a toxic prion peptide (PrP 106-126) triggers the secretion of a neuronal death signal, which may be reactive oxygen species. Neurons in culture are differentially susceptible to death based upon their expression of PrPC: wild-type neurons are killed by peptide-activated microglial cells, whereas neurons cultured from PrP −/− knockout mice are partially protected. Again, it appears that the expression of PrP^{C} is essential for the transduction of a cell death signal for neurons. Astrocytes, a proliferation-competent cell in the brain, can also be activated by PrP^{Sc} (DeArmond et al, Prog. Brain Res. 94:437-46 (1992)). Another proliferation-incompetent cell is the mature muscle fiber. Prusiner and colleagues (Westaway et al, Cell 76(1):117-29 (1994)) have described a degenerative muscle disease in transgenic mice overexpressing of PrP^{C}, again suggesting that PrP^{C} may participate in the transduction of a death signal.

In lymphocytes, a peripheral proliferation-competent cell, PrP^{C} apparently participates in cell activation. Cashman and colleagues (Cashman et al, Cell 61:185-92 (1990)) have shown that lymphocyte PrP^{C} is dramatically upregulated by cell activation, and antibodies directed against PrP^{C} suppress T cell activation induced by the lectin mitogen conconavalin A. Bruce and colleagues (Mabbott et al, Immunology 92(2):161-5 (1997)) have also shown that PrP −/− knockout mice display defects in activation with the lectin mitogen conconavalin A. It is possible that the cell activation signal provided by PrP^{C} to proliferation-competent cells such as lymphocytes and astrocytes might be the same signal that induces cell death in proliferation-incompetent cells such as neurons and myotubes. The scientific literature would support the idea that apoptosis in neurons may be due to the ectopic activation of genes involved in cell cycle progression.

These data suggest that the normal and abnormal signalling of PrP^{C} may be pharmacologically manipulated as a novel target to control cell activation in disorders of abnormal or undesirable activation and proliferation. Since the normal signalling of PrP^{C} must at least in part be regulated by an interaction with its cell surface receptor, agents which modify this interaction may prove useful in the treatment of these disorders which include, but are not limited to, cancer, autoimmune disease, and graft rejection.

PrP-AP

The construction of a baculoviral construct for the production of a prion protein (PrP) and alkaline phosphatase (AP) fusion protein in insect cells, as well as the production of the cellular supernatant containing this fusion protein have been described in WO97/45746 published on Dec. 4, 1997.

PrP-AP Binding to Cell Surfaces

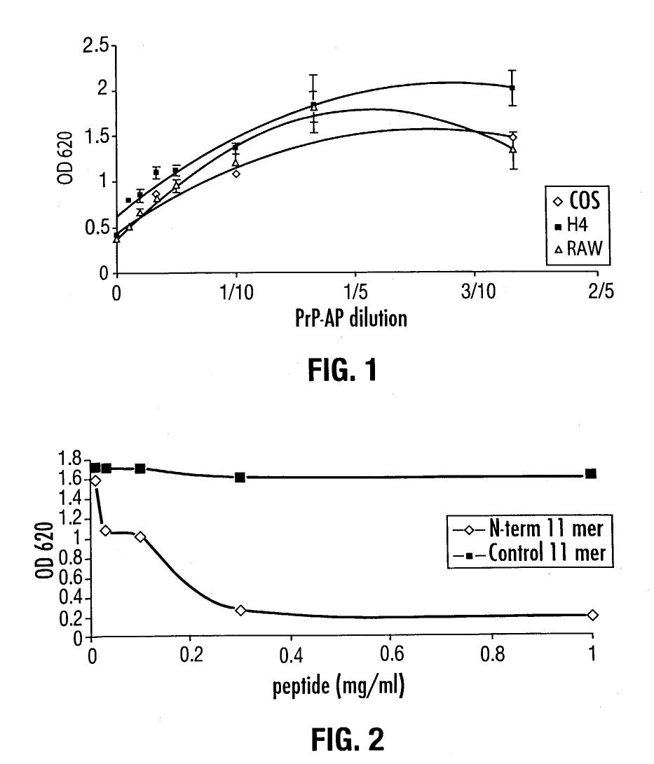

In WO97/45746 above the characteristics of PrP-AP binding to cell surfaces were shown. In the present application we verified these properties on the cell lines to be used subsequently. These cell lines were: COS-7 (ATCC CRL-1651), H4 (ATCC HTB-148) and RAW 264.7 (ATCC TIB-71). These cells were grown to confluency in 96 well plates (Costar™ cat #3526), usually overnight, in DMEM (Cellgro™) supplemented with 10% fetal bovine serum (Wisent). Each well was then washed with 200 ml HBHA (1× Hank's salt solution, 0.5 mg/ml bovine serum albumin (BSA), 20 mM HEPES buffer pH 7, and 0.1% sodium azide). After washing, the cells were incubated with PrP-AP containing supernatant at varying dilutions (⅓, 1/10 and 1/30 for COS cells and ⅓, ⅙, 1/10, 1/20, 1/30, 1/50 and 1/100 for H4 and RAW cells). All dilutions were done using Dulbecco's phosphate buffered saline (PBS; Cellgro) with 1 mM CaCl₂. The PrP-AP supernatant used throughout these experiments was produced on Sep. 22, 1999 and frozen at −80° C. in aliquots. Incubation proceeded for 1.5 hours at room temperature. After the incubation the cells were washed six times the same way as above and then lysed with 55 ml per well of lysis buffer (50 mM tris buffer pH 8, 150 mM NaCl, 0.02% sodium azide and 1% triton X-100). The lysis proceeded 15 minutes at room temperature and 12 minutes at 65° C. to inactivate heat labile endogenous phosphatase activities. The 96 well plate was then centrifuged at 1500 rpm (™ GP8R centrifuge) for 5 minutes. 50 ml of lysate per well were transferred to a new plate. The remaining heat stable alkaline phosphatase activity was assayed by adding 150 ml of Blue Phos™ phosphatase substrate (Kierkegaard and Perry Laboratories) and reading the optical density (OD) at a wavelength of 620 nm after 1-1.5 hour incubation at room temperature. PrP-AP bound the surfaces of all three cell lines to an equivalent degree (FIG. 1).

Inhibition of PrP-AP Binding by a PrP Peptide

From preliminary evidence it was proposed that the amino terminal of the PrP moiety of PrP-AP was responsible for the binding to cell surfaces. To test this hypothesis some peptides with the amino terminal PrP sequences were produced and included in the PrP-AP binding assay described above. The peptides (synthesized by Phoenix Pharmaceuticals, Inc.; PrP 11 amino terminal amino acid sequence (N-term 11 mer): KKRPKPGGWNT (SEQ ID NO: 5)); control sequence (control 11 mer): GGSRYPGQGSP (SEQ ID NO: 6)) were dissolved in Dulbecco's PBS at a concentration of 10 mg/ml. From this stock quantities were added to a PrP-AP containing supernatant diluted 1/10 in PBS to obtain concentrations of 1 mg/ml, 0.3, 0.1, 0.03, 0.01 mg/ml. Only the peptide with the PrP amino terminal sequence inhibited the binding of PrP-AP to cell surfaces (FIG. 2).

Inhibition of Binding by Copaxone

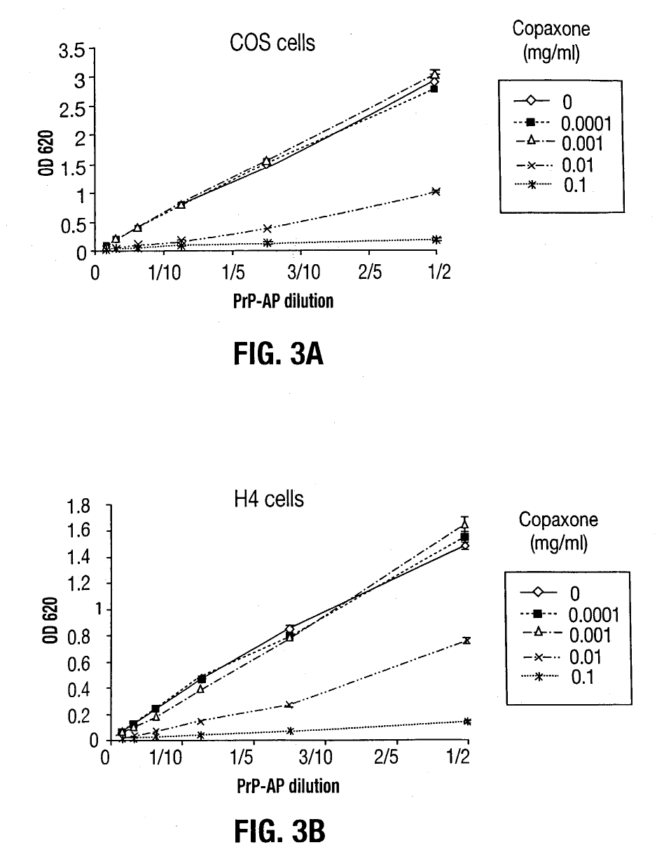

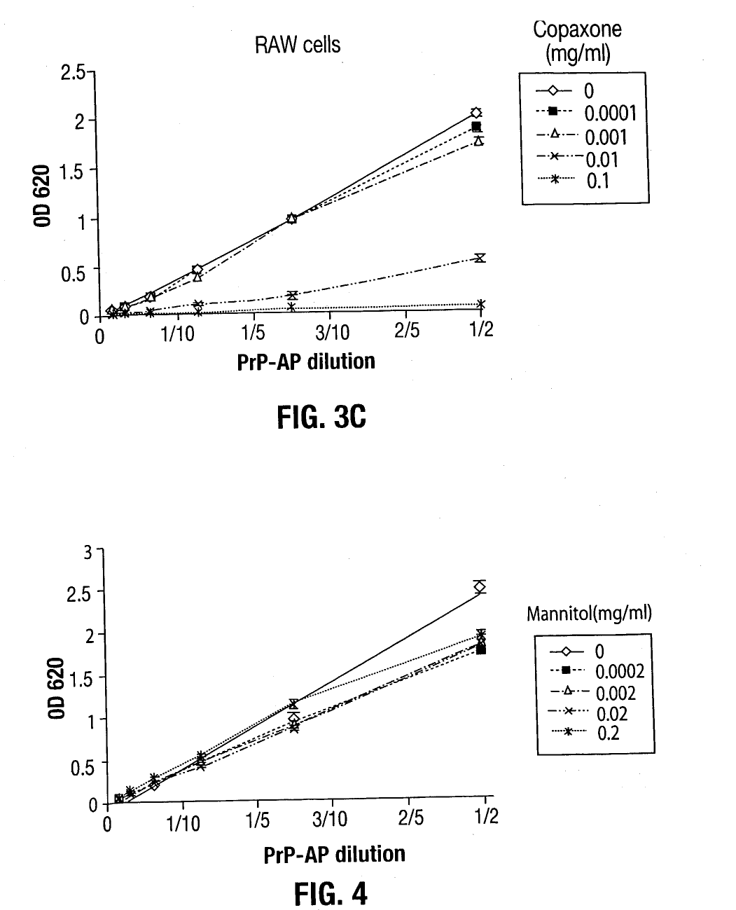

In order to assess the effects of Copaxone on the binding of PrP-AP to cell surfaces the assays from above were repeated with Copaxone added to the PrP-AP containing supernatant dilutions. A certain amount of copaxone was weighed and dissolved in PBS with 1 mM CaCl₂ to a concentration of 1 mg/ml (not including the weight of mannitol present in the Copaxone formulation). From this initial solution quantities were added to PrP-AP containing supernatant dilutions in order to achieve concentrations of 0.1 mg/ml, 0.01, 0.001 and 0.0001 mg/ml. All incubations and washes were done exactly as above. Copaxone inhibited PrP-AP binding to all three cell lines (FIG. 3).

In order to show that the effect seen with Copaxone is due to the copaxone copolymer content as opposed to the mannitol content the same assays were performed using mannitol alone instead of Copaxone. Mannitol was dissolved in PBS with 1 mM CaCl₂ at a concentration of 2 mg/ml (the same concentration that can be found in the 1 mg/ml Copaxone solution above since for each mg of copaxone copolymer there are 2 mg of mannitol in the formulation) and used the same way as the Copaxone solution was above.

For the three cell lines the results looked like the example in FIG. 4 and illustrate that mannitol does not participate in the inhibition of binding by Copaxone.

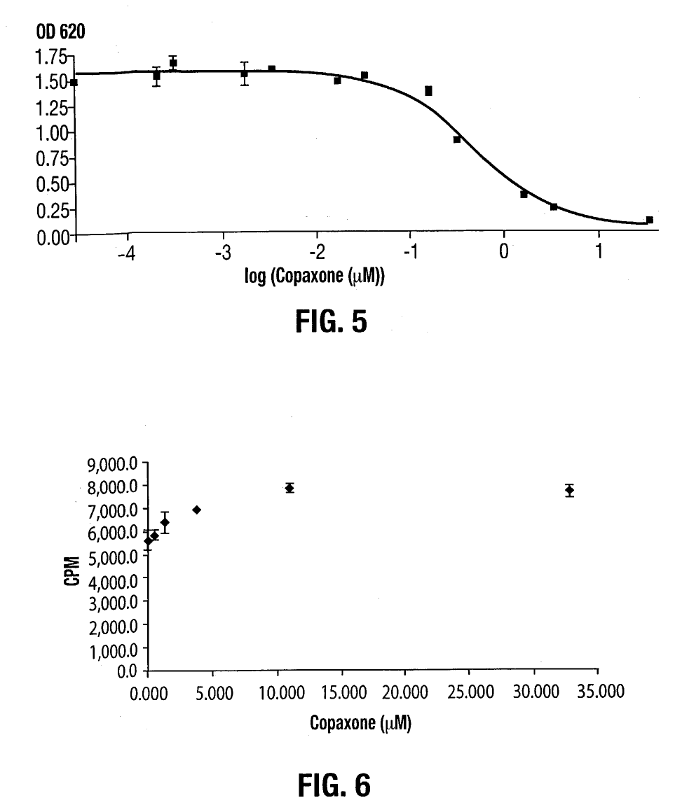

A more detailed dose-response curve was produced for both COS and H4 cells in which PrP-AP was kept constant at a 1/10 dilution and Copaxone was varied from 0.1 nM to 100 mM (molarity was calculated by using a mean molecular weight of 7.85 kDa for a Copaxone batch with molecular weights ranging from 4.7 kDa to 11 kDa). From this study the half-maximal inhibitory concentration (IC50) can be estimated at 0.5 mM (FIG. 5).

Specificity of Copaxone Inhibition for PrP-AP Binding

To verify that Copaxone specifically inhibits PrP-AP binding to cell surfaces two alternative receptor-ligand pairs were tested. Iodinated epidermal growth factor ([125I]-EGF) binds cell surfaces via an EGF-receptor (E. Adamson and A. Rees, Mol. And Cell. Biochem., vol. 34, pp.129-152, 1981). H4 cells, which were shown to express the EGF receptor by flow cytometry analysis, were cultured to 70% confluency in 24 well dishes (Costar) in DMEM (Cellgro) supplemented with 10% fetal bovine serum (Wisent) usually overnight. These cells were then washed twice with Dulbecco's PBS (Cellgro) supplemented with 0.1% bovine serum albumin (BSA; Sigma). The cells were incubated 90 minutes on ice with 1 nM [125I]-EGF which was diluted from an 85 nM stock (Pharmacia Amersham) in Dulbecco's PBS supplemented as above. To this [125I]-EGF solution varying concentrations of Copaxone were added (33 mM, 11, 3.7, 1.2 and 0.41 mM). After the incubation the cells were washed three times with the supplemented PBS and then lysed with 500 ml 1N sodium hydroxide. This lysate was transferred to vials and counted for gamma radiation. The presence of Copaxone did not affect the capacity of EGF to interact with the EGF receptor on cell surfaces (FIG. 6).

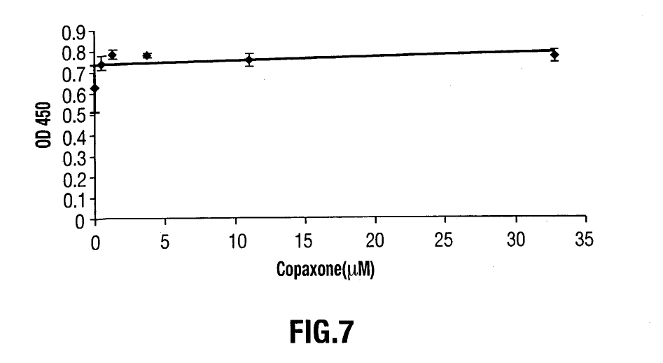

Transferrin binds the transferrin receptor on the surface of many growing cells. Transferrin was labelled with biotin using biotindisulfide N-hydroxysuccinimide ester (Sigma biotinylation kit). After dialysis to eliminate unreacted biotin, this stock concentration was of 30 mM. The presence of the receptor on H4 cells was verified by flow cytometry analysis. These cells were then cultured in 10 cm dishes (Nunc) to 70% confluency in DMEM supplemented with fetal bovine serum and then processed according to a published protocol (A. Vieira, Molecular Biotechnology, vol. 10, pp. 247-250, 1998). First the cells were washed three times with buffer A (20 mM HEPES, 100 mM potassium acetate, 85 mM sucrose, 1 mM magnesium acetate, pH 7.4), scraped from the dishes into a 50 ml centrifuge tube, centrifuged at 1000 rpm in a Centra GP8R™ centrifuge at 4° C. for 5 minutes and resuspended in one pellet volume of buffer B1 (20 mM HEPES, 100 mM potassium acetate, 0.75 mM sucrose, 1 mM magnesium acetate, pH 7.4). The cells were then stored at −80° C. taking note of the total number of cells per aliquot. Prior to an experiment the cells were thawed, centrifuged in a microcentrifuge (IEC micromax™) at maximal speed and resuspended in buffer B2 (buffer B1 without sucrose) so that 300,000 cells are contained in 10 ml. To 10 ml of cells, 29 ml of biotinylated transferrin were added at a final dilution of 1/1000 in binding buffer (20 mM HEPES, 100 mM NaCl, 0.1% BSA, pH 7.5) and 1 ml of a 2% BSA solution. This binding mixture was incubated 3.5 hours at room temperature. The cells were then washed three times with 1 ml binding buffer. 100 ml of ST buffer (1 mM EDTA, 50 mM NaCl, 10 mM Tris-HCl, 0.1% SDS, 1% Triton X-100TM, 0.2% BSA, pH 7.4) were added to the cells. This cell lysate was placed onto a well of a 96 well plate (Nunc poly-sorp™) which had been coated with anti-transferrin antibody (DAKO) and the volume in each well was brought to 200 ml with ST buffer. The plate was incubated at 4° C. overnight and then washed twice with PBS, once with ST buffer and three more times with PBS. Bound biotinylated transferrin was detected by incubating with peroxidase conjugated streptavidin (Boehringer Mannheim) at a dilution of 1/10,000 for 50 minutes at room temperature followed by washes in the same sequence as above and incubation with a peroxidase substrate (BM-blue POD substrate; Boehringer Mannheim). After stopping the reaction with 1M H₂SO₄ the result was read at 450 nm using a plate reader (Spectramax™). The presence of Copaxone had no effect on biotinylated transferrin binding (FIG. 7).

Copolymer Specificity

Since Copaxone is a simple copolymer of four amino acids, other copolymer compounds can be used to decipher what components are important to its action. A selection of these copolymers that ranged in size and amino acid composition was tested for effects on PrP-AP binding to cell surfaces. These compounds were used in the same PrP-AP containing supernatant binding assay conditions as the experiments described in the section above. A list of the copolymers tested is shown in table 1.

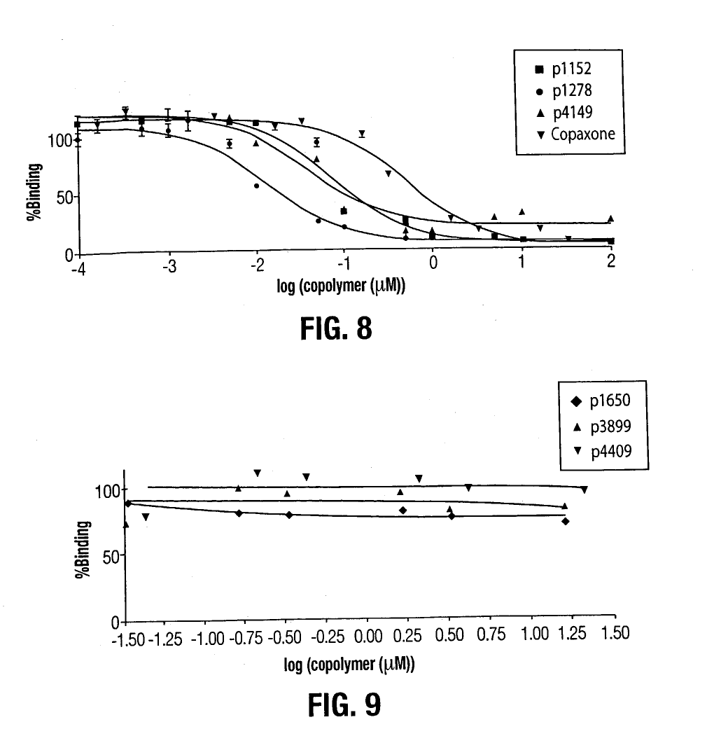

These compounds vary by size, overall charge and amino acid composition but always close to the Copaxone composition. Of these compounds those that have the same composition as Copaxone were inhibitory (p1152 and p1278) as were p4149, p4274 and p4659. Although the mechanism of inhibition was not fully explored there seems to be a dependence on charge and molecular weight. FIG. 8 is an experiment that compares Copaxone to p1152, p1278 and p4149. Clearly all compounds are inhibitory and the biggest copolymer, p1278 at 52 kDa, is the most effective followed by p1152 and p4149 that have intermediate molecular weights of 24.8 kDa and 28.9 kDa and finally Copaxone, the lightest of the compounds at an average molecular weight of 7.85 kDa (FIG. 8). The next figure (FIG. 9) shows an experiment with copolymers that contain more negative charges: p1650, p3899 and p4409. None of these copolymers inhibit the binding of PrP-AP to cell surfaces.

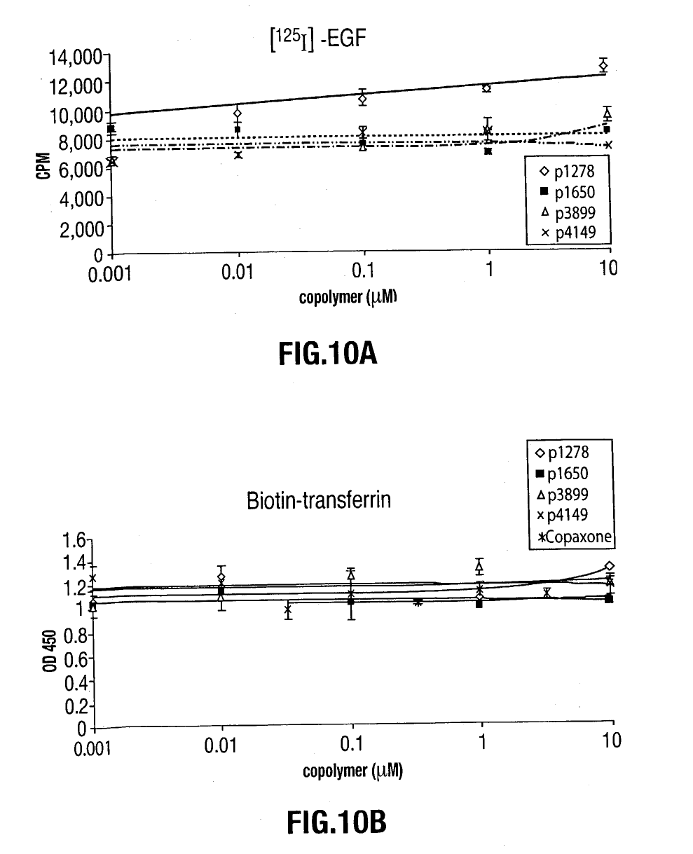

Compounds of different charge and size characteristics were also tested in the [125I]-EGF assay and the biotinylated transferrin assay. None of the copolymers were inhibitory to the binding of these ligands to their cell surface receptors (FIG. 10).

It is expected that copolymers of the invention comprising at least K, R and P will be of particular value in combatting prion-related diseases or inhibiting binding of prion proteins to cell surfaces. The amino acids K, R and P can be in any order, number and position in the copolymer though the KKRPK (SEQ ID NO: 1) sequence is expected to be of particular interest. The KKRPK (SEQ ID NO: 1) sequence need not be at the end of the molecule but can be elsewhere as long as it can be “presented”. For ease of synthesis the KKRPK (SEQ ID NO: 1) sequence can be attached at one end or the other of the molecule. Longer parts of the N-terminal sequence of PrP are also of interest and use as parts of copolymers of the invention and, likewise, component amino acids of the N-terminal sequence can be presented in an order different from the order found at the N-terminal of PrP.

An anti-prion therapeutic according to the invention may be administered with a pharmaceutically-acceptable diluent, carrier, or excipient, in unit dosage form. For example, conventional pharmaceutical practice may be employed to provide suitable formulations or compositions to administer such ahti-prion therapeutics to animals suffering from or presymptomatic for a prion disease, or at risk for developing a prion disease. Any appropriate route of administration can be employed, for example, parenteral, intravenous, subcutaneous, intramuscular, intracranial, intraobital, ophthalmic, intraventricular, intracapsular, intraspinal, intracisternal, intraperitoneal, intranasal, aerosol, or oral administration.

The therapeutic composition of the invention may comprise only the anti-prion therapeutic as an active ingredient or is more often administered with such a diluent, carrier or excipient as mentioned above. The amount of active ingredient may range by weight from 1-99%, preferably 5-95%, particularly 10-90% of the composition. Particular composition may comprise 20-80%, 30-70%, 40-60% or even 50% active ingredient.

Methods well known in the art for making formulations are found in, for example, “Remington's Pharmaceutical Sciences”. Formulations for parenteral administration can, for example, contain excipients, sterile water, or saline, polyalkylene glycols such as polyethylene glycol, oils of vegetable origin, or hydrogenated naphthalenes. Biocompatible, biodegradable lactide polymer, lactide/glycolide copolymer, or polyoxyethylene-polyoxypropylene copolymers can be used to control the release of the compounds. Other potentially useful parenteral delivery systems for anti-prion therapeutic compounds include ethylene-vinyl acetate copolymer particles, osmotic pumps, implantable infusion systems, and liposomes. Formulations for inhalation can contain excipients, for example, lactose, or can be aqueous solutions containing, for example, polyoxyethylene-9-lauryl ether, glycocholate and deoxycholate, or can be oily solutions for administration in the form of nasal drops, or as a gel.

The methods of the present invention may be used to reduce or prevent the disorders described herein in any animal, for example, humans, domestic pets, or livestock. Where a non-human animal is treated, the anti-prion therapeutic employed is preferably specific for that species.

TABLE 1
Selection of copolymers (Sigma)

| | | | | | Molecular |
|---|---|---|---|---|---|
| Copolymer | Sequence | Ratio | Charges | | weight |
| Copaxone | Y, E, A, | 1.0:1.4: | 5.2 | pos., | 4.7 to 11 Kda. |
| | K | 3.4:4.2 | 1.4 | neg. | |
| Sigma | Y, E, A, | 1:2:6:5 | 6 | pos., | 24.8 Kda. |
| p1152 | K | | 2 | neg. | |
| Sigma | Y, E, A, | 1:2:6:5 | 6 | pos., | 52 Kda. |
| p1278 | K | | 2 | neg. | |
| Sigma | E, A, Y | 1:1:1 | 1 | pos., | 28.9 Kda. |
| p4149 | | | 1 | neg. | |
| Sigma | E, A, Y | 6:3:1 | 6 | neg., | 30.6 Kda. |
| p3899 | | | 1 | pos. | |
| Sigma | E, K, Y | 6:3:1 | 6 | neg., | 23.3 Kda. |
| p4409 | | | 4 | pos. | |
| Sigma | K, Y | 1:1 | 2 | pos. | 128 Kda. |
| p4274 | | | | | |
| Sigma | K, Y | 4:1 | 5 | pos. | 24.6 Kda. |
| p4659 | | | | | |
| Sigma | K, Y | 1:9 | 10 | pos. | 80 Kda. |
| p2025 | | | | | |
| Sigma | E, A | 6:4 | 6 | neg. | 30 Kda. |
| p1650 | | | | | |

A = alanine
E = glutamate
K = lysine
Y = tyrosine

What is claimed is:

1. A pharmaceutical composition that inhibits binding of prion protein to a cell surface or combats a prion-related disease comprising a positively charged copolymer including a basic amino acid selected from the group consisting of lysine and arginine in association with a pharmaceutically acceptable diluent or carrier.

2. A composition according to claim 1 wherein said copolymer has a net positive charge and additionally comprises at least one amino acid selected from the group consisting of glutamate, alanine and tyrosine.

3. A composition according to claim 1 wherein said copolymer additionally comprises glutamate and alanine.

4. A composition according to claim 1 wherein said copolymer has a molecular weight of from 5 to 130 Kilodaltons.

5. A composition according to claim 1 wherein said copolymer comprises copaxone.

6. A pharmaceutical composition according to claim 1 wherein said copolymer comprises lysine (K), arginine (R) and proline (P).

7. A pharmaceutical composition according to claim 6 wherein lysine, arginine and proline are present in the ratio 3 lysine:1 arginine:1 proline.

8. A pharmaceutical composition according to claim 6 wherein lysine, arginine and proline are present in the ratio 3 lysine:1 arginine:2 proline.

9. A pharmaceutical composition according to claim 6 wherein said copolymer additionally comprises glycine and lysine, arginine, proline and glycine are present in the ratio 3 lysine:1 arginine:2 proline:1 glycine.

10. A pharmaceutical composition according to claim 6 wherein said copolymer additionally comprises glycine and lysine, arginine, proline and glycine are present in the ratio 3 lysine:1 arginine:2 proline:2 glycine.

11. A pharmaceutical composition according to claim 10 wherein said copolymer additionally comprises tryptophan and lysine, arginine, proline, glycine and tryptophan are present in the ratio 3 lysine:1 arginine:2 proline:2 glycine:1 tryptophan.

12. A pharmaceutical composition according to claim 11 wherein said copolymer additionally comprises asparagine and lysine, arginine, proline, glycine, tryptophan and asparagine are present in the ratio 3 lysine:1 arginine:2 proline:2 glycine:1 tryptophan:1 asparagine.

13. A pharmaceutical composition according to claim 12 wherein said copolymer additionally comprises threonine and lysine, arginine, proline, glycine, tryptophan, asparagine and threonine are present in the ratio 3 lysine:1 arginine:2 proline:2 glycine:1 tryptophan:1 asparagine:1 threonine.

14. A pharmaceutical kit for combatting a prion-related disease comprising a positively charged copolymer including a basic amino acid selected from the group consisting of lysine and arginine, a pharmaceutically acceptable diluent or carrier and instructions for use in combatting said disease.

15. A kit according to claim 14 wherein said copolymer additionally comprises at least one amino acid selected from the group consisting of glutamate, alanine and tyrosine.

16. A kit according to claim 14 wherein said copolymer additionally comprises glutamate and alanine.

17. A kit according to claim 14 wherein said copolymer has a molecular weight of from 4 to 130 Kilodaltons.

18 A kit according to claim 14 wherein said copolymer comprises copaxone.

19. A kit according to claim 14 wherein said copolymer comprises lysine (K), arginine (R) and proline (P).

20. A kit according to claim 19 wherein lysine, arginine and proline are present in the ratio 3 lysine:1 arginine:1 proline.

21. A kit according to claim 19 wherein lysine, arginine and proline are present in the ratio 3 lysine:1 arginine:2 proline.

22. A kit according to claim 19 wherein said copolymer additionally comprises glycine and lysine, arginine, proline and glycine are present in the ratio 3 lysine:1 arginine:2 proline:1 glycine.

23. A kit according to claim 20 wherein said copolymer additionally comprises glycine and lysine, arginine, proline and glycine are present in the ratio 3 lysine:1 arginine:2 proline:2 glycine.

24. A kit according to claim 23 wherein said copolymer additionally comprises tryptophan and lysine, arginine, proline, glycine and tryptophan are present in the ratio 3 lysine:1 arginine:2 proline:2 glycine:1 tryptophan.

25. A kit according to claim 24 wherein said copolymer additionally comprises asparagine and lysine, arginine, proline, glycine, tryptophan and asparagine are present in the ratio 3 lysine:1 arginine:2 proline:2 glycine:1 tryptophan:1 asparagine.

26. A kit according to claim 25 wherein said copolymer additionally comprises threonine and lysine, arginine, proline, glycine, tryptophan, asparagine and threonine are present in the ratio 3 lysine:1 arginine:2 proline:2 glycine:1 tryptophan:1 asparagine:1 threonine.

27. A method of preparing a pharmaceutical composition in ready-to-use form for combatting a prion-related disease, which comprises admixing a copolymer as defined in claim 1 with a pharmaceutically acceptable diluent or carrier.
